(19) United States
(12) Patent Application Publication
(10) Pub. No.: US 2004/0138586 A1
(43) Pub. Date: Jul. 15, 2004

(54) Apparatus and method for determining yield pressure

(76) Inventors: Robert A. Ganz, Minnetonka, MN (US); Mark Rydell, Golden Valley, MN (US)

Correspondence Address: Schwegman, Lundberg, Woessner & Kluth, P.A., P.O. Box 2938, Minneapolis, MN, 55402, US

(21) Appl. No.: 10/718,268

(22) Filed: Nov. 20, 2003

Related U.S. Application Data
(60) Provisional application No. 60/427,783, filed on Nov. 20, 2002.
(60) Provisional application No. 60/456,194, filed on Mar. 20, 2003.
(60) Provisional application No. 60/472,938, filed on May 23, 2003.
(60) Provisional application No. 60/502,996, filed on Sep. 15, 2003.

(51) Int. Cl. A61B 5/00

(52) U.S. Cl. 600/560

(57) ABSTRACT

An apparatus includes a catheter for insertion into a stomach to introduce air into the stomach through the catheter and a pressure measurement device associated with the catheter to sense a pressure in the stomach. The pressure measurement device is used to determine a gastric yield pressure of the stomach when enough air has been introduced to the stomach to overcome the esophageal-gastric junction.

### RELATED APPLICATIONS

This application claims the benefit under 35 U.S.C. 119(e) of U.S. Provisional Application Serial No. 60/427,783 filed Nov. 20, 2002, U.S. Provisional Application Serial No. 60/456,194 filed Mar. 20, 2003, U.S. Provisional Application Serial No. 60/472,938 filed May 23, 2003, and U.S. Provisional Application Serial No. 60/502,996 filed Sep. 15, 2003, the specifications of which are incorporated herein by reference.

### FIELD OF THE INVENTION

This invention relates to an apparatus and method for measuring gastric yield pressure and distal esophageal compliance.

### BACKGROUND OF THE INVENTION

Gastroesophageal reflux disease (GERD) is an epidemic disease and one of the major health problems in the world. It affects approximately 60 million people in the United States alone, with 20 million of those having daily symptoms. It is one of the most common causes of gastrointestinal related disability, is responsible for billions of dollars in healthcare costs, and is responsible for multiple serious potential complications. Gastroesophageal reflux is the proximate cause of Barrett's esophagus, and is the main cause of esophageal carcinoma in the United States and developed world.

Although research in the area of GERD has been intense, it is still unclear why some people develop this condition and others do not. There is a “reflux barrier zone”, also known as the lower esophageal sphincter (LES) zone, located at the esophageal-gastric junction (EGJ), that prevents acid reflux in health. Disturbances and malfunction of this reflux barrier zone, predisposes to GERD. The barrier zone is comprised of several components including, LES pressure, LES length, anatomical elements such as the presence or absence of a hiatal hernia, and lower esophageal compliance or gastric yield pressure.

Gastric yield pressure is the pressure within the stomach at which gastric content, including gastric acid, is able to be forced through the reflux barrier zone and through the esophageal-gastric (EG) junction, into the esophagus to cause acid reflux. As the distal esophagus becomes increasingly more compliant and less “stiff”, a condition that occurs with aging and chronic overdistention of the stomach from overeating, the gastric yield pressure required to overcome the EG junction barrier falls making one more susceptible to acid reflux. These alterations in esophageal compliance and yield pressure, with or without alterations and disturbances in LES pressure, LES length and anatomy, contribute to malfunctioning of the reflux barrier zone, causing chronic GERD.

It is therefore desirable to be able to measure gastric yield pressure, and distal esophageal compliance, in order to diagnose GERD or the predisposition to GERD, and also to measure the impact of surgical or endoscopic therapies designed to treat GERD. There are multiple surgical and endoscopic interventions that treat GERD, at least in part, by improving (decreasing) distal esophageal compliance and thereby increasing the gastric yield pressure necessary to cause reflux. These include, but are not limited to, Nissen fundoplication, placement of an Angelchik prosthesis, endoscopic sewing such as the Bard Endocinch procedure, endoscopic radio-frequency treatment of the LES such as the Stretta procedure, and biopolymer implantation via either intraluminal bulking with the Medtronic Gatekeeper prosthesis, or via intramural implantation via the Boston Scientific Enteryx device.

### SUMMARY

Accordingly, it would be beneficial to be able to measure the impact of these therapies on gastric yield pressure, to determine whether patients have been adequately treated or require further interventions. Moreover, it would be advantageous if yield pressure and esophageal compliance could be measured during endoscopy, a diagnostic test of the esophagus, due to ease of use. It would also be useful if the yield pressure measuring device could fit through the endoscope, in order to decrease patient discomfort, and to avoid having patients swallow an additional device or have a device passed trans-nasally.

The present system provides an apparatus and method for the measurement of gastric yield pressure and distal esophageal compliance. In one embodiment, the apparatus and method can include a catheter for insertion into the lumen of the stomach, a hollow portion of the catheter for infusing air into the stomach, a pressure transducer associated with the catheter to record pressure measurements, an air infusing mechanism at the proximal end positioned outside of the patient's body, and a recording device for monitoring and reading out the gastric pressure.

In some embodiments, the yield pressure catheter can be inserted via use of an endoscope; the yield pressure catheter can be inserted into a preexisting channel in the endoscope, and then advanced into the stomach; the yield pressure catheter can be attached to the outside of an endoscope and then advanced into the stomach; or the catheter can be attached to the outside of an endoscope via adhesive, glue, pre-fit molded attachment, or clip.

In some embodiments, the catheter is not hollow, but can be a solid-state recording device, with a separate air infusing mechanism or tube, or the catheter can be a water-perfused system, with a proximal water-containing chamber for perfusion and pressure measurement.

In some embodiments, the catheter can be advanced into the stomach under direct visualization; or the catheter can be advanced over a guide-wire without direct visualization, or without endoscopic guidance, either with or without x-ray or fluoroscopic guidance. In one example, when passed without endoscopic guidance, the catheter can be solid, or a solid-state pressure device, with at least one transducer incorporated into its shaft, either at the tip of the catheter or elsewhere. In one example, when passed without endoscopic guidance, the catheter can be a water-perfused system, with a proximal water-containing chamber for perfusion and measurement.

In some embodiments, the catheter can be placed trans-nasally or trans-orally with or without a guide-wire and with or without x-ray or fluoroscopic guidance. The catheter can have a detachable pressure transducer. In one example, the catheter can have a detachable pressure transducer, which can be temporarily attached to the inner gastric lining to record pressure, and which can then be detached or sloughed into the lumen of the stomach. In a further embodiment, the detachable pressure transducer, temporarily attached to the inner gastric lining, can record pressure and wirelessly transmit said information to a receiving and recording device located outside the body.

In one embodiment, the catheter can be hollow or solid, or solid-state with a hollow portion for air insufflation, or with at least one pressure transducer built into the shaft either at the tip or elsewhere. The air infusing device can be a hand pump, pneumatic pump, balloon, or bag. In one embodiment, the monitoring and reading device can be analog or digital.

In one embodiment, the transducer can be attached to the distal portion of the catheter by adhesive, glue, pre-fit molded attachment, or clip.

In one example use, the monitoring catheter and transducer do not significantly affect the lumen of the EG junction, in order to achieve a more accurate gastric yield pressure recording. In one embodiment, the endoscope does not advance across the EG junction, but the yield pressure catheter is advanced across the EG junction.

In one embodiment, the system includes an apparatus and method for recording gastric yield pressure and distal esophageal compliance in a patient with or without upper gastrointestinal tract disease, with or without GERD.

In one embodiment, the system includes an apparatus and method for recording gastric yield pressure and distal esophageal compliance in a patient before or after a surgical, medical or endoscopic intervention such as Nissen fundoplication, Angelchik prosthesis placement, Bard Endocinch procedure, Stretta procedure, intraluminal bulking such as with beads or the Medtronic Gatekeeper prosthesis, or intramural implantation of bio-polymers such as the Boston Scientific Enteryx procedure.

In one embodiment, the system includes an apparatus and method for the measurement of gastric yield pressure and distal esophageal compliance. The apparatus and method can include a catheter for insertion into the stomach, a hollow portion of the catheter for infusion of air into the stomach, a hollow chamber in the catheter for conducting the pressure via a common cavity from the stomach to a proximal recording device, a pressure transducer located at the proximal portion of the catheter for accurate pressure measurement, and an air infusing mechanism at the proximal end of the catheter.

In one embodiment, the catheter can be a solid-state recording catheter, with or without a separate mechanism for infusing air into the stomach, with at least one transducer incorporated into its shaft, either at the tip of the catheter or elsewhere. In one embodiment, the catheter can be a water-perfused system, with a proximal water-containing chamber for perfusion and measurement.

The present system provides an apparatus and method for accurate measurement of the gastric yield pressure and esophageal compliance, to aid in the diagnosis and treatment of gastro-esophageal reflux disease, and other disorders of the upper gastrointestinal tract. In one or more embodiments, the system provides a yield pressure-measuring device that can be used in conjunction with an endoscope, and more specifically, pass through the biopsy channel of the endoscope, or be attached to the endoscope, to facilitate its use.

These and other more detailed advantages of the present invention will be better understood by reference to the following figures and detailed description, which illustrate by way of example of but a few of the various forms of the invention within the scope of the appended information.

### BRIEF DESCRIPTION OF FIGURES

FIGS. 1A and 1B show a portion of the upper gastrointestinal tract.

FIG. 2 shows a yield pressure measuring apparatus according to one embodiment.

FIG. 3 shows a yield pressure measuring apparatus according to one embodiment.

FIG. 4 shows a yield pressure measuring apparatus according to one embodiment.

FIG. 5 shows a yield pressure measuring apparatus according to one embodiment.

FIG. 6 shows a yield pressure measuring apparatus according to one embodiment.

FIG. 7A shows a yield pressure measuring apparatus according to one embodiment.

FIG. 7B shows a cross-sectional view of a catheter according to one embodiment.

FIG. 8 shows a yield pressure measuring apparatus according to one embodiment.

FIG. 9 shows a yield pressure measuring apparatus according to one embodiment.

FIG. 10A shows a yield pressure measuring apparatus according to one embodiment.

FIG. 10B shows a cross-section view of a catheter according to one embodiment.

FIG. 11 shows a sectional side view of a catheter according to one embodiment.

FIG. 12 shows a cross-sectional view of a catheter according to one embodiment.

### DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense.

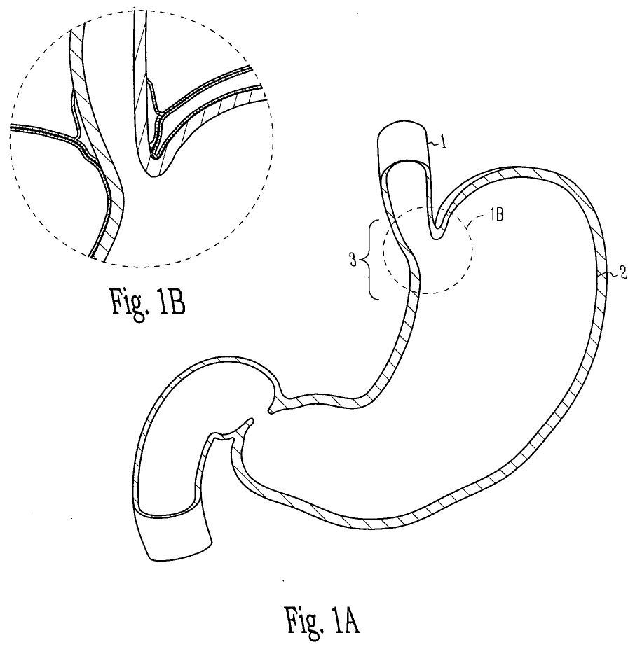

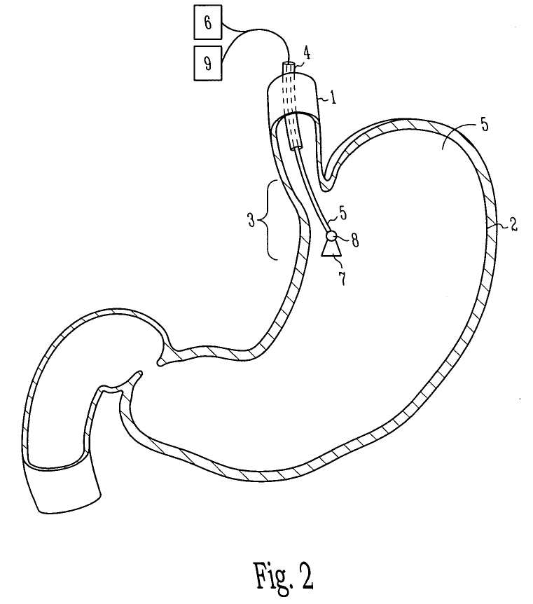

Some embodiments of the inventive subject matter disclosed herein concern a method and apparatus for determining and measuring the gastric yield pressure and esophageal compliance in the area of the EG junction. FIGS. 1A and 1B show a portion of the upper gastrointestinal tract, including the esophagus 1, the stomach 2, and the area between the esophagus and stomach, the EG junction 3. The EG junction is considered to be the reflux barrier zone, and is comprised of the distal few centimeters of the esophagus and the proximal few centimeters of the stomach. This area is also called the lower esophageal sphincter zone.

In health, the area of the EG junction is typically closed to prevent reflux of acid and other gastric content into the esophagus. This area will transiently open for a few seconds to allow the passage of food, but otherwise remains sealed effectively separating the esophagus and the stomach into two separate cavities, with different pressures. Typically, in health, the pressure in the esophagus is −5 mm Hg, the pressure in the stomach is +5 mm Hg, and the pressure at the EG junction, or LES pressure is +15-20 mm Hg. Thus, the normally functioning reflux barrier or LES, at the EG junction, keeps the two organs (esophagus and stomach) separate.

It is known that increasing gastric pressure, which occurs when the stomach starts to fill with food, causes opening stress on the area of the EG junction. At some point, the pressure in the stomach becomes so high, that it forces open the area of the EG junction from below, overriding the high pressure zone of the reflux barrier, and causing the two previously separate cavities to become a single common cavity. The point at which this opening occurs, is called the gastric yield pressure, because the EG junction “yields” to increased gastric pressure. At this point, gastric acid and other content refluxes into the esophagus, causing GERD. Also when this occurs, the stomach decompresses because content has escaped into the esophagus, pressure in the stomach falls, and the cavities again become separated due to the higher EG junction pressure. Typically then, the highest measurable gastric pressure is the yield pressure.

The yield pressure is inversely proportional to the compliance of the distal esophagus. In health, the distal esophagus tends to have low compliance which will generate a relatively higher yield pressure, whereas someone with GERD may have a relatively highly compliant distal esophagus which will be noted as a lower yield pressure.

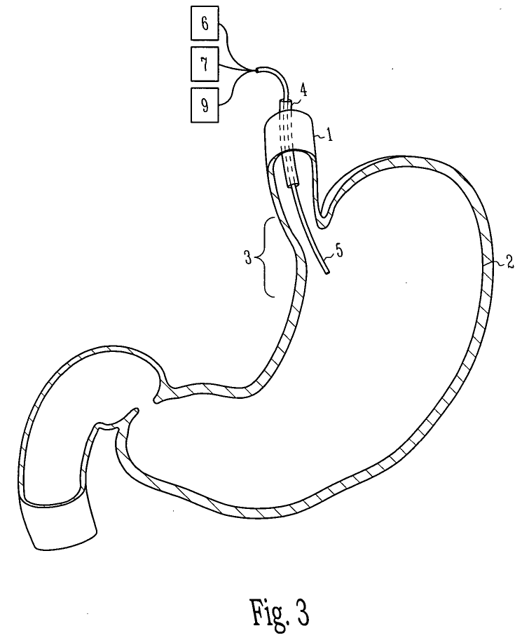

FIG. 2 shows a yield pressure measuring apparatus according to one embodiment. An endoscope 4 is positioned in the usual manner, known in the field of gastroenterology, through the mouth and into the distal esophagus 1 proximal to the EG junction 3, which is readily identified. A yield pressure catheter 5 is then inserted into a channel in the endoscope and then maneuvered across the EG junction 3 and into the proximal portion of the stomach 2, under direct visualization, in a manner well-known to the art of catheter placement. It is important that the endoscope not cross the EG junction 3 which might then affect the opening pressure. In one embodiment, the yield pressure catheter is hollow and can insufflate air via its hollow construction into the stomach 2. The air is introduced through the catheter via a pump 6 which is external to the patient. Other types of air insufflation devices can also be used including a balloon, a hand pump, a pneumatic bag, etc.

The air is pumped into the stomach and the resultant rise in gastric pressure is measured via a pressure measurement device, such as a pressure transducer 7 attached to the distal portion of the catheter 5, which is residing in the stomach 2. The transducer 7 can be attached to the distal portion of the catheter by a clip 8, in any manner of ways, either by adhesive or glue, or via a pre-fit molded attachment that fits onto the tip of the endoscope. As the pressure transducer measures the steadily rising pressure, the information reads out on a monitor 9 attached to the catheter. The monitor 9 information read out could be in an analog fashion as well as a digital fashion. The peak pressure is noted at the time the EG junction opens and the gastric pressure begins to fall.

Refer now to FIG. 3. In this embodiment the yield pressure catheter 5 is placed into the stomach as described above, but in this example the hollow catheter has no transducer at its distal tip. Rather the hollow catheter is used to pump air into the stomach via pump 6, for example. The catheter conducts the pressure through its hollow chamber back to a pressure transducer 7 at the proximal portion of the catheter, outside the body. In this example, both the transducer 7 and the recording device or monitor 9 are positioned at the proximal end of the catheter. The transducer 7 measures the pressure in the stomach via the common cavity created by having an open tube in the stomach chamber.

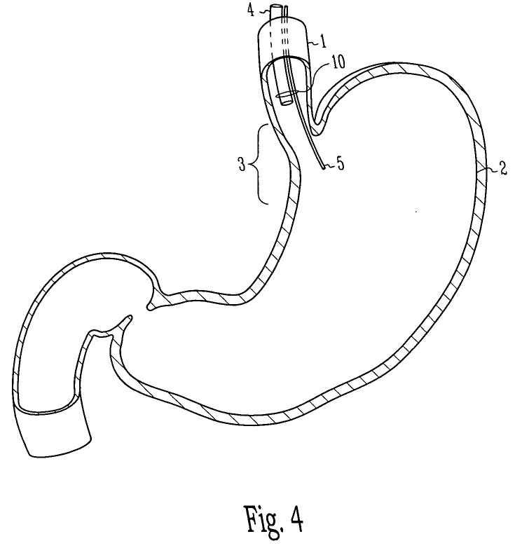

Refer now to FIG. 4. In this embodiment the yield pressure catheter 5 is attached to the outside of the endoscope, rather than through an inner endoscope channel. The catheter 5 can be attached in any manner of ways i.e. via adhesive, glue, a clip device 10, pre-fit molded attachment etc. Any clip or pre-fit molded device could also be detachable, and pass through the gastrointestinal tract of the patient following placement of the catheter. The attachment to the external position of the endoscope is to facilitate placement through the mouth and into the distal esophagus. Once appropriately positioned, the catheter 5 can be advanced or retracted as needed through the EG junction 3 and into the proximal stomach for recording of the yield pressure, in any manner as described above in FIGS. 2 and 3, or as described below.

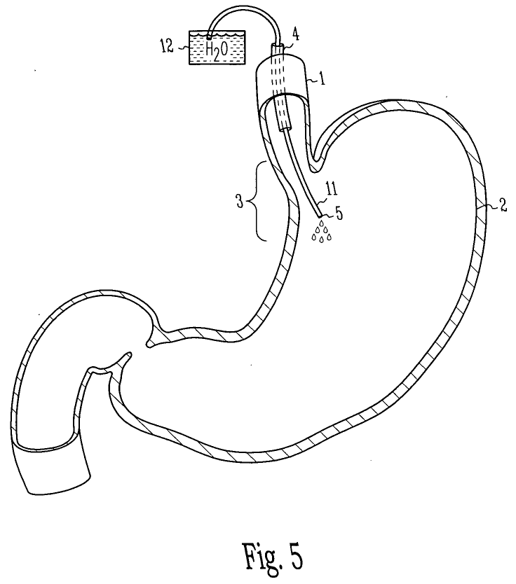

In some embodiments, the yield pressure catheter 5 need not be hollow, but rather could be a solid-state recording device. For example, the yield pressure catheter can have at least one pressure measurement device such as a transducer incorporated into its shaft at the tip or also in other locations. The catheter could also contain a separate mechanism or tube or lumen for infusing air. In some embodiments, as shown in FIG. 5, the catheter 5 need not infuse air, rather the catheter could be water-perfused 11, and have an attached water chamber 12 at its proximal portion for perfusion and pressure measurement.

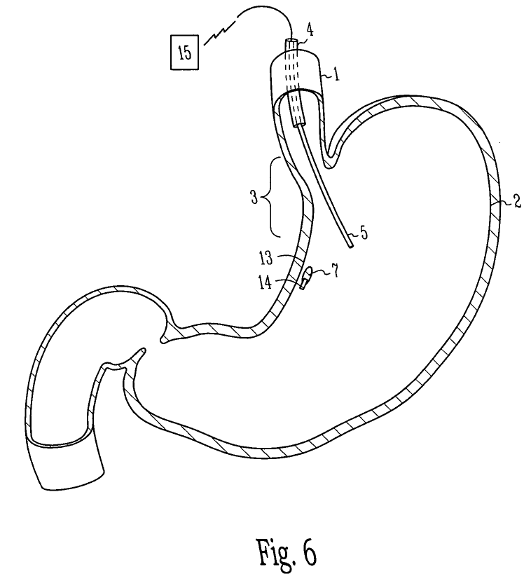

Refer now to FIG. 6. In this embodiment the yield pressure catheter 5 is advanced either through the endoscope, or attached to the outside of the endoscope for positioning as before in the proximal stomach. The pressure transducer 7 in this example is detachable, and is left in place in the proximal stomach. The attachment is made to the inner lining of the stomach, i.e. the mucosal lining 13, by a pin 14. The attachment could also be made in any manner of ways e.g. adhesive, glue, suction, sutures, clips etc. In one example of this embodiment the stomach is distended as before, from air insufflation through the catheter, while the detachable transducer records the yield pressure. In another example, the pressure transducer is left in place for hours or days, and continuously records the yield pressure as the patient eats several meals, and distends the stomach naturally. The transducer then spontaneously detaches from the gastric wall. In this example the pressure recordings are transmitted wirelessly via a radio transmitter to a receiving device 15 outside the body.

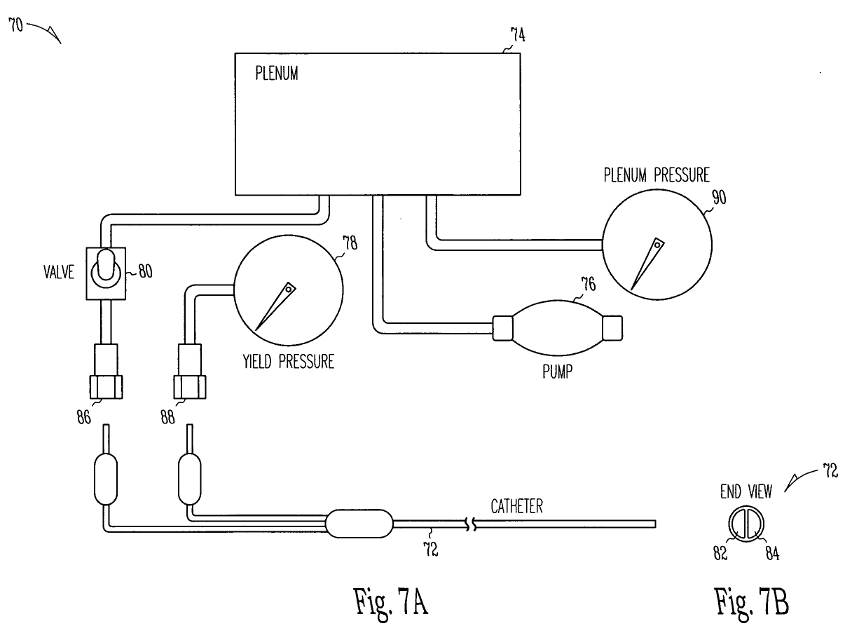

FIG. 7A shows a yield pressure measurement system 70, according to one embodiment. System 70 includes a double lumen catheter 72 (see FIG. 7B), and an air plenum 74 which is pressurized using a pump 76, such as a hand pump, for example. In this example, a pressure-measuring device such as a gauge 78 is at the proximal end of the catheter, not inside the patients stomach.

One embodiment utilizes plenum or reservoir 74, which can be pressurized using hand pump 76, for example in the form of a rubber bulb. When the position of a valve 80 is changed to open, the air is released in a steady, even flow. The use of double lumen catheter 72 allows for the air to be injected into the stomach through one lumen 82, and the pressure inside the stomach to be measured through a separate lumen 84. An advantage of this of this is that the pressure sensor is isolated from the injection means, and therefore pressure spikes associated with injecting air into the stomach are eliminated. The increase in stomach pressure is displayed on the gauge 78 as an even sweep of the needle, and it is quite easy therefore to identify the point at which air starts escaping, or the yield pressure. Additional air can be added to the plenum 74 at any time without causing pressure spikes on the gauge 78. In one embodiment, the plenum 74 can be eliminated with the pump 76 pumping air directly into the stomach, and the pressure reading would still be accurate and spike free. However, plenum 74 allows for a steady and even build-up of pressure in the stomach.

In one example use of system 70, catheter 72 is passed through the working channel of a scope, as discussed above. Under direct visualization the catheter 72 is advanced into the stomach, and then the scope is retracted so that the catheter resides in the stomach and the scope is in the esophagus. To attach the catheter to the yield pressure device, connect the two terminal plugs into the appropriate fitting (86, 88) on the faceplate. The toggle valve 80 is placed in the closed position and actuating the pump 76 pressurizes the plenum 74. The plenum pressure can be monitored by use a pressure gauge 90 connected to the plenum. When ready, air is released from the plenum 74 by changing the position of the toggle valve 80 to open. The pressurized air will begin to flow out of the plenum and into the stomach via one of the two lumens in the catheter. The yield pressure gauge 78 is in communication with the stomach via the other lumen in the catheter, so as pressure builds in the stomach it is recorded on the gauge. At any time, additional air pressure can be added to the plenum; in fact it is desirable to keep that pressure at about 200 mm of Hg. The yield pressure can be determined by observing the maximum reading achieved on the yield pressure gauge 78 as the stomach fills with air. If an analog gauge is used, a maximum-indicating pointer can be incorporated into the gauge to record the highest pressure reached.

In one embodiment, a pressure transducer could also be used instead of analog gauge 78. An advantage of this would be that the transducer output could be recorded over time. When connected to a digital display, for example by connecting to a lap top computer with appropriate software, a graph could be created and later printed and permanently stored. By using a constant rate of air injection and a transducer, it is possible that a value could be obtained that represents the period of time between when the sphincter opens and when it reseals. In other words, the time interval between the pressure drop and the resumption of pressure building again. This may be of some diagnostic value.

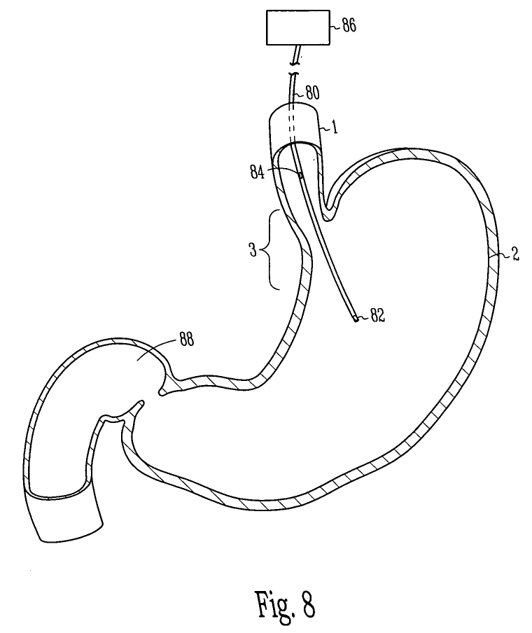

FIG. 8 shows a yield pressure catheter 80 according to one embodiment. In this example, yield pressure catheter 80 includes a distally located pressure measurement device such as a pressure transducer 82 and a pressure measurement device such as a pressure transducer 84 located on an intermediate portion of catheter 80. Pressure transducer 84 is located such that it is in the esophagus 1 above the EG junction 3 when pressure transducer 82 is in stomach 2. Transducers 82 and 84 are operatively coupled to a monitor 86 so the pressures at each can be noted or recorded, in a similar fashion as discussed above. In this example, when the EG junction 3 yields to the increased gastric pressure, the pressures at transducers 82 and 84 should be similar due to the common cavity effect caused by the opening of the EG junction. Such a configuration prevents a false diagnosis of the gastric yield pressure which can occur if the increased gastric pressure is relieved through duodenum 88 instead of EG junction 3. In other embodiments, a multi-lumen catheter can be used having a first opening distally located to measure the gastric pressure and a mid-catheter opening to measure the esophageal pressure, in a manner as described above.

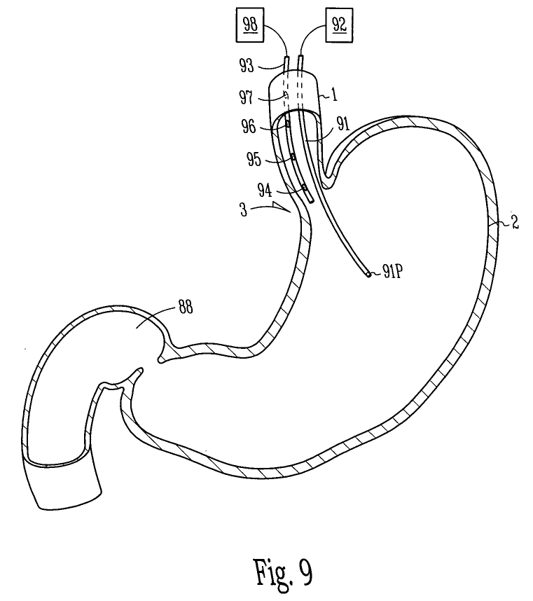

FIG. 9 shows a yield pressure measuring system 90 according to one embodiment. System 90 is another example of a yield pressure measurement system configured to prevent a false diagnosis of the gastric yield pressure which can occur if the increased gastric pressure is relieved through duodenum 88 instead of EG junction 3. System 90 includes a yield pressure measurement catheter 91 coupled to a monitor 92. Catheter 91 can include any of the yield pressure measurement catheters discussed above, and can include a pressure measurement device such as a transducer 91P at a distal end, for example.

In one embodiment, system 90 also includes a water perfused manometry catheter 93 coupled to a perfusion pump/monitor 96. Two or more sensor openings 94, 95, 96, and 97 are located on catheter 93. Catheter 93 is placed in the esophagus 1 with sensors 94-97 running along a portion of the esophagus and into the EG junction 3. In use, air is delivered to the stomach via catheter 91 in a manner as described above. When a pressure drop is sensed in the stomach by transducer 91P, the sensor openings 94-97 record simultaneous waveforms indicating the common cavity effect of the esophageal sphincter opening. This indicates that there is a yield at the EG junction as opposed to duodenal yield. If there are not simultaneous waveforms, then a duodenal yield has occurred. Examples of water perfused catheters 93 include a Dentsleeve water perfused catheter and a Synectics water perfused manometry catheter of Medtronic. In some embodiments, a catheter having solid state sensors can also be used to detect the common cavity effect.

In one embodiment, a yield pressure measurement catheter can be provided which measures both the yield pressure via a transducer, such as transducer 91P, and also detects if the common cavity effect occurs, as discussed above.

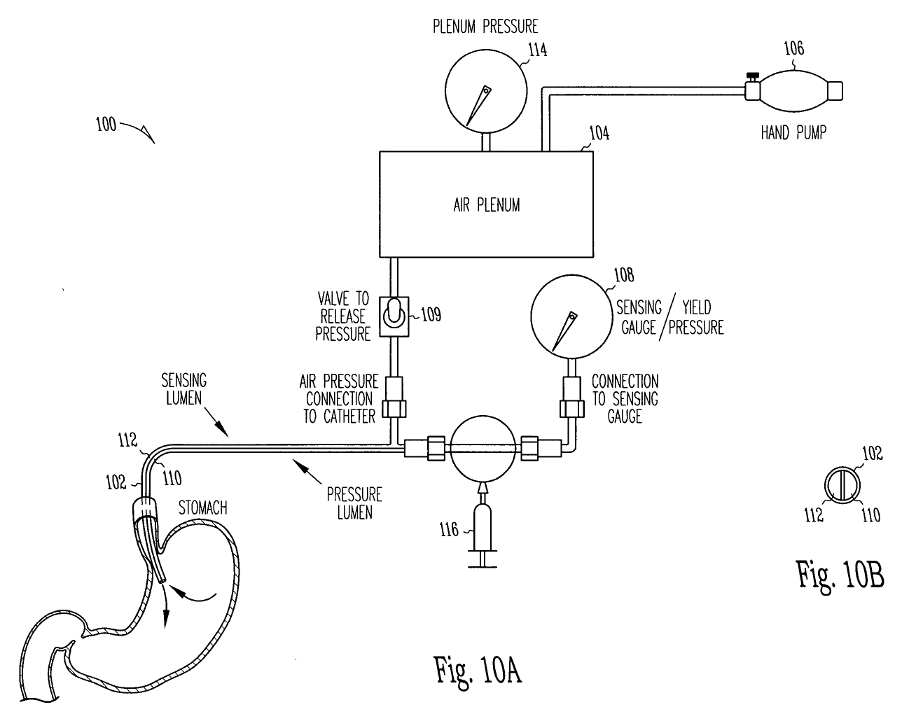

FIG. 10A shows a yield pressure measuring system 100 according to one embodiment. System 100 includes a double lumen catheter 102, and an air plenum 104 which is pressurized using a pump 106, such as a hand pump, for example. In this example, a pressure-measuring device 108 is at the proximal end of the catheter, not inside the patients stomach.

One embodiment utilizes plenum or reservoir 104, which can be pressurized using hand pump 106, for example in the form of a rubber bulb. When the position of a valve 109 is changed to open, the air is released in a steady, even flow. The use of double lumen catheter 102 allows for the air to be injected into the stomach through one lumen 110, and the pressure inside the stomach to be measured through a separate lumen 112. An advantage of this of this is that the pressure sensor is isolated from the injection means, and therefore pressure spikes associated with injecting air into the stomach are eliminated. The increase in stomach pressure is displayed on the gauge 108 as an even sweep of the needle, and it is quite easy therefore to identify the point at which air starts escaping, or the yield pressure. Additional air can be added to the plenum 104 at any time without causing pressure spikes on the gauge 108. In one embodiment, the plenum 104 can be eliminated with the pump 106 pumping air directly into the stomach, and the pressure reading would still be accurate and spike free. However, plenum 104 allows for a steady and even build-up of pressure in the stomach.

In one example use of system 100, catheter 102 is passed through the working channel of a scope, as discussed above. Under direct visualization the catheter 102 is advanced into the stomach, and then the scope is retracted so that the catheter resides in the stomach and the scope is in the esophagus. The catheter is connected to the plenum and pressure sensing gauge as described above for system 70. The toggle valve 109 is placed in the closed position and actuating the pump 106 pressurizes the plenum 104. The plenum pressure can be monitored by use a pressure gauge 114 connected to the plenum. When ready, air is released from the plenum 104 by changing the position of the toggle valve 109 to open. The pressurized air will begin to flow out of the plenum and into the stomach via one of the two lumens in the catheter. The yield pressure gauge 108 is in communication with the stomach via the other lumen in the catheter, so as pressure builds in the stomach it is recorded on the gauge. At any time, additional air pressure can be added to the plenum; in fact it is desirable to keep that pressure at about 200 mm of Hg. The yield pressure can be determined by observing the maximum reading achieved on the yield pressure gauge 108 as the stomach fills with air. If an analog gauge is used, a maximum-indicating pointer can be incorporated into the gauge to record the highest pressure reached.

In this embodiment, a syringe 116, or other pressure means can be coupled to sensing lumen 112. Syringe 116 is used to blow out lumen 112 if the lumen becomes clogged on the distal end.

Referring to FIG. 10B, in one embodiment the double lumens of catheter 102 can have unequal diameters. For example, the inflow or pressure lumen 110 can be ⅓ of the total diameter of the catheter and the outflow or sensing lumen 112 can be ⅔ of the total diameter. Such a structure also helps prevent the sensing lumen from being clogged or blocked.

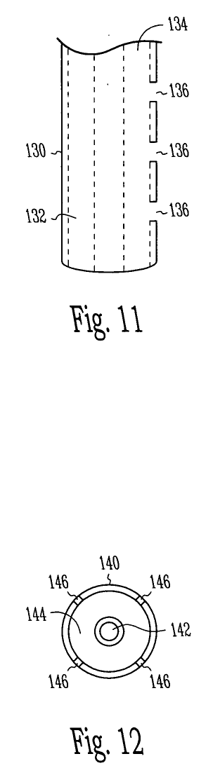

FIG. 11 shows a double lumen catheter 130, which can be used in accordance with one embodiment. Catheter 130 includes an outflow/pressure lumen 132 and an inflow/sensing lumen 134. Lumen 134 includes one or more ports 136 proximate the distal end of the lumen. Again, these ports 136 help prevent clogs or a blocking of lumen 134. The distal end of the catheter can therefore be submerged in fluid, and as long as one of ports 136 remains open, fluid will not be forced into the air-sensing lumen 134.

FIG. 12 shows a cross section view of a double lumen catheter 140, according to one embodiment. This design is similar to catheter 130 except catheter 140 is coaxial. The inner lumen 142 is the one that feeds the air into the stomach. The space between the tubes (i.e. the space between outside surface of inner tube and inside surface of outer tube) is a lumen 144 that sends air pressure back to the gauge. The outer lumen 144 includes one or more ports 146 proximate the end. This design allows ports 146 be all around the tube.

It is noted that any of the above embodiments of the device can be also introduced into the stomach, without the aid of the endoscope, i.e. introduced separately from any other visualizing device or any other guidance device. It is also noted that the yield pressure catheter can be passed over a guide-wire, into the stomach with or without x-ray or flouroscopic guidance. In these situations the yield pressure catheter can be passed either trans-nasally or trans-orally.

It is also noted that in the situation of passage without an endoscope, the pressure transducer can be detachable, can attach to the inner lining of the stomach by clip, pin, glue, adhesive, etc. and can wirelessly transmit pressure information to a recording device outside the body.

It is also noted that in the situation of passage without an endoscope, the catheter does not have to be hollow, it can be solid with or without a hollow portion for air infusion, a solid-state recording device with or without a separate mechanism or tube for air infusion, or a water-perfused system with a proximal water-containing chamber for perfusion and pressure measurement.

It is understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

What is claimed is:

1. An apparatus comprising:
a catheter for insertion into a stomach to introduce air into the stomach through the catheter; and
a pressure measurement device associated with the catheter to sense a pressure in the stomach, wherein the pressure measurement device is used to determine a gastric yield pressure of the stomach when enough air has been introduced to the stomach to overcome the esophageal-gastric junction.

2. The apparatus of claim 1, wherein the pressure measurement device includes a pressure transducer at a distal end of the catheter.

3. The apparatus of claim 1, wherein the pressure measurement device includes a pressure transducer at a proximal end of the catheter to measure a pressure of the air in the catheter.

4. The apparatus of claim 1, wherein the catheter includes at least two lumens, a first lumen for introducing air into the stomach and a second lumen associated with the pressure measurement device to measure the pressure in the stomach.

5. The apparatus of claim 4, wherein the two lumens have unequal diameters, with the second lumen having a larger diameter than the first lumen.

6. The apparatus of claim 4, wherein the second lumen includes one or more side ports open to the stomach.

7. The apparatus of claim 4, wherein the lumens are co-axial lumens.

8. The apparatus of claim 1, further comprising a second pressure measurement device associated with the catheter, wherein the second pressure measurement device is located so as to measure a pressure in the esophagus when the first pressure measurement device measures the pressure in the stomach.

9. The apparatus of claim 8, wherein the catheter comprises a water-perfused catheter having one or more sensor openings along an intermediate portion of the catheter and a pressure transducer at a distal end of the catheter.

10. A system comprising:
a catheter to introduce air into a stomach through the catheter;
a pressure measurement device to sense a pressure in the stomach; and
a monitor operatively coupled to the pressure measurement device to display the pressure of the stomach, wherein the monitor reveals a gastric yield pressure of the stomach when enough air has been introduced to the stomach by the catheter such that the pressure in the stomach overcomes an esophageal-gastric junction.

11. The system of claim 10, further comprising a second pressure measurement device to measure a pressure in an esophagus when the first pressure measurement device measures the pressure in the stomach.

12. The system of claim 10, wherein the monitor includes a pressure gauge.

13. The system of acclaim 10, wherein the pressure measurement device includes a detachable pressure transducer attached to a wall of the stomach.

14. The system of claim 10, wherein the catheter includes at least two lumens, a first lumen for introducing air into the stomach and a second lumen associated with the pressure measurement device to measure the pressure in the stomach.

15. The system of claim 14, wherein the two lumens have unequal diameters, with the second lumen having a larger diameter than the first lumen.

16. The system of claim 10, including an endoscope to introduce the catheter into the stomach.

17. A method comprising:
inserting a distal end of a catheter into a stomach, the catheter associated with a pressure measurement device;
introducing air into the stomach through the catheter; and
determining a gastric yield pressure using the pressure measurement device when enough air has been introduced to the stomach to overcome the esophageal-gastric junction.

18. The method of claim 17, wherein the catheter includes at least two lumens, a first lumen for introducing air into the stomach and a second lumen coupled to the pressure measurement device.

19. The method of claim 17, further comprising a second pressure measurement device associated with the catheter, wherein the second pressure measurement device is located for measuring a pressure in the esophagus when the first pressure measurement device measures the pressure in the stomach.

20. The method of claim 17, wherein the catheter is inserted into the stomach using an endoscope.
